(19) United States
(12) Patent Application Publication
(10) Pub. No.: US 2023/0190731 A1
(43) Pub. Date: Jun. 22, 2023

(54) FORMULATION COMPRISING HIF PROLYL HYDROXYLASE INHIBITORS

(71) Applicant: ZYDUS LIFESCIENCES LIMITED, Ahmedabad, Gujarat (IN)

(72) Inventors: M.E. KANNAN, Ahmedabad, Gujarat (IN); Mukesh UKAWALA, Ahmedabad, Gujarat (IN); Ritu LADDHA, Ahmedabad, Gujarat (IN); Amit PRAJAPATI, Ahmedabad, Gujarat (IN); Jitendra PATEL, Ahmedabad, Gujarat (IN)

(21) Appl. No.: 17/912,228

(22) PCT Filed: Mar. 17, 2021
(86) PCT No.: PCT/IB2021/052214

§ 371 Date: Sep. 16, 2022

(30) Foreign Application Priority Data
Mar. 17, 2020 (IN) 202021011431

(51) Int. Cl. A61K 31/4709; A61K 9/48; A61K 9/20; A61K 9/28

(52) U.S. Cl. CPC A61K 31/4709; A61K 9/485; A61K 9/2009; A61K 9/2013; A61K 9/2027; A61K 9/2054; A61K 9/2813; A61K 9/2853; A61K 9/2866; A61K 9/4825; A61K 9/4858; A61K 9/4866

(57) ABSTRACT

The present invention generally relates to a pharmaceutical composition of suitable HIF prolyl hydroxylase inhibitors. Preferably, the present invention discloses novel formulations of the compound of formula (Ia), or pharmaceutically acceptable salts of compounds of formula (Ia). More particularly the present invention relates to the pharmaceutical composition of compounds of formula (Ia) comprising compounds of formula (Ia) or its pharmaceutically acceptable salts.

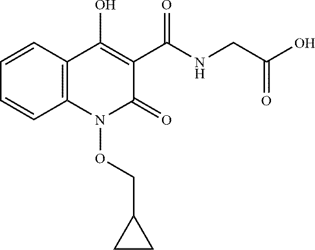 [SMILES: O=C(O)CNC(=O)c1c(O)c2ccccc2n(OCC2CC2)c1=O]

### FIELD OF THE INVENTION

The present invention generally relates to a pharmaceutical composition of suitable HIF prolyl hydroxylase inhibitors. Preferably, the present invention discloses novel formulations of the compound of formula (Ia), or pharmaceutically acceptable salts of compounds of formula (Ia). More particularly the present invention relates to the pharmaceutical composition of compounds of formula (Ia) comprising compounds of formula (Ia) or its pharmaceutically acceptable salts.

### BACKGROUND OF THE INVENTION

Hypoxia-inducible factor (HIF) is a heteroduplex, with α and β subunit. The beta subunit is usually present in excess, while the alpha subunit is the limiting factor in the formation of the functional dimer. The HIF-α subunit binds with the β subunit in the nucleus and, with the cooperation of cofactors, binds to DNA sequences called hypoxia response elements, and hence induces expression of target genes. There are three isoforms of the α subunit, HIP-1α, HIF-2α and HIF-3α. The activity of HIF is regulated via hydroxylation at two proline residues by an oxygen-sensitive family of prolyl hydroxylase enzymes (PHD), known as PHD1, PHD2 and PHD3. Hydroxylation at one or both of these proline residues allows binding of HIF-α first by the von Hippel--Lindau tumor suppressor protein (pVHL) and then by ubiquitin ligase which results in rapid ubiquitination and proteosomal degradation. The HIF-α subunits are also regulated by hydroxylation at a C-terminal asparagine residue by factor inhibiting HIF (FIH), an oxygen-dependent hydroxylase enzyme. Factor inhibiting HIF prevents the recruitment of transcriptional coactivators, thereby blocking the activity of HIF.

WO2014102818 discloses compounds of the following general formula

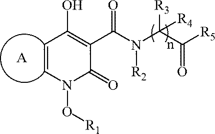

These compounds are reported to be useful for the treatment of anemia. It has been surprisingly found that the compound of formula (Ia) is a potent inhibitor of hypoxia inducible factor (HIF) prolyl hydroxylase. HIF prolyl hydroxylase inhibitors are useful for increasing the stability and/or activity of HIF, and useful for, inter alia, treating and preventing disorders associated with HIF, including anemia, and ischemia- and hypoxia-related disorders.

The structural formula of compounds of formula (Ia) is shown below.

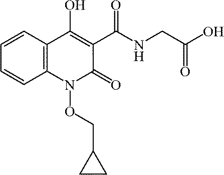 [SMILES: O=C(O)CNC(=O)c1c(O)c2ccccc2n(OCC2CC2)c1=O]

The compounds of formula (Ia) are insoluble in 0.1 N HCL, partially soluble in water, soluble in alkaline aqueous condition and freely soluble in N,N-dimethyl formamide.

### SUMMARY OF THE INVENTION

The present invention, describes a pharmaceutical composition of compounds of formula (Ia) or its pharmaceutically acceptable salts.

### EMBODIMENT OF THE INVENTION

In one embodiment, the present invention provides a pharmaceutical composition of compound of Formula (Ia) or its pharmaceutical acceptable salt.

In another embodiment, the present invention provides a pharmaceutical composition of compound of formula (Ia) optionally with other suitable pharmaceutical excipients.

In another embodiment, the present invention provides an uncoated tablet comprising compounds of formula (Ia) or its pharmaceutical acceptable salts and a pharmaceutically acceptable excipient.

In a further embodiment, the present invention provides a coated tablet comprising compounds of formula (Ia) or its pharmaceutical acceptable salts and a pharmaceutically acceptable excipient. In one embodiment, the tablet comprises a tablet core and a coating.

In another embodiment, the present invention provides a capsule comprising compound of formula (Ia) or its pharmaceutically acceptable salts and a pharmaceutical acceptable excipient. In one embodiment, the capsule comprises a capsule fill and capsule shell.

In one embodiment, the capsule fill comprises compound of formula (Ia) and the pharmaceutically acceptable excipient.

In a further embodiment, the present invention provides a process for the preparation of pharmaceutical formulation of compound of formula (Ia) or its pharmaceutically acceptable salts thereof.

In yet another embodiment, a method for treating, pre-treating, or delaying onset or progression of a condition mediated at least in part by hypoxia inducible factor (HIF) is provided. The method comprises administering to a patient in need thereof, a pharmaceutical formulation, a tablet, or a capsule as described herein.

In still yet another embodiment, a method for treating, pre-treating, or delaying onset or progression of anaemia is provided. The method comprises administering to a patient in need thereof, a pharmaceutical formulation, a tablet, or a capsule as described herein.

### DETAILED DESCRIPTION OF THE INVENTION

The present invention describes a pharmaceutical composition of compounds of formula (Ia)

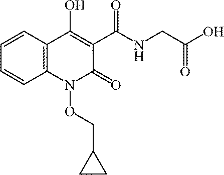 [SMILES: O=C(O)CNC(=O)c1c(O)c2ccccc2n(OCC2CC2)c1=O]

or its pharmaceutically acceptable salts.

The present invention further describes a pharmaceutical composition of compounds of formula (Ia) or its pharmaceutical acceptable salts, comprising one or more pharmaceutical excipients.

In one embodiment, the pharmaceutical composition of the present invention comprise compound of formula (Ia) or its pharmaceutically acceptable salts having particle size distributions, wherein the compound of formula (Ia) or its pharmaceutically acceptable salts have D₉₀ value of not more than 450 microns.

In one embodiment pharmaceutical excipients according to present invention can be selected from solubilizers, diluents or fillers, disintegrants, binder, lubricants, glidants, film forming agents, plasticizers, opacifier, solvents, and the like as known in the art.

In one embodiment, the present invention provides an uncoated tablet comprising compounds of formula (Ia) or its pharmaceutical acceptable salts and a pharmaceutically acceptable excipient.

In a further embodiment, the pharmaceutically acceptable excipient in the uncoated tablet comprises microcrystalline cellulose, starch, croscarmellose sodium, lactose monohydrate, hypromellose, polyvinyl pyrrolidone, colloidal silicon dioxide, talc and magnesium stearate.

In an another embodiment, the uncoated tablet comprises from about 1% to about 90% w/w compound of formula (Ia); microcrystalline cellulose from about 2% to about 90% w/w; croscarmellose sodium from about 0.5% to 10% w/w; lactose monohydrate from about 2% to about 90% w/w; hypromellose 3 cps from about 0.5% to about 10% w/w; Talc from about 0.5% to about 3% w/w; magnesium Stearate from about 0.5% to about 5% w/w of the total composition, polyvinyl pyrolidone from about 0.5% to about 10% w/w; starch from about 1% to about 20% w/w based on the weight of uncoated tablet.

In one embodiment, the present invention provides a coated tablet comprising compound of formula (Ia) or its pharmaceutical acceptable salts and a pharmaceutically acceptable excipient.

In a further embodiment, the pharmaceutically acceptable excipient in the coated tablet comprises microcrystalline cellulose, starch, croscarmellose sodium, lactose monohydrate, hypromellose polyvinyl pyrrolidone, colloidal silicon dioxide, talc and magnesium stearate. In a further embodiment, the pharmaceutically acceptable excipients for coating comprises hyperomellose, polyvinyl alcohol, polyethylene glycols and titanium dioxide or suitable coating ready materials selected from Opadry.

In another embodiment, the coating is present in the tablet in an amount that is from about 0.5% to about 5% w/w of hypromellose 3cps; polyethylene glycol from about 0.25% to about 1.0% w/w; titanium dioxide from about 0.25% to about 2.0% w/w or the tablets can also be coated using the readily available coating material like Opadry, wherein the amount is from about 0.5% to about 5.0% w/w based on the weight of the tablet core.

In another embodiment, the tablet core comprises from about 1% to about 90% w/w compound of formula (la); microcrystalline cellulose from about 2% to about 90% w/w; croscarmellose sodium from about 0.5% to 10% w/w; lactose monohydrate from about 2% to about 90% w/w; hypromellose 3 cps from about 0.5% to about 10% w/w; talc from about 0.5 to about 5% w/w; magnesium stearate from about 0.5% to about 3% w/w based on the weight of coated tablet.

In one embodiment, the present invention provides a capsule comprising compound of formula (Ia) or its pharmaceutical acceptable salts and a pharmaceutically acceptable excipient.

In a further embodiment, the pharmaceutically acceptable excipient in the capsule comprises microcrystalline cellulose, starch, mannitol, lactose monohydrate, croscarmellose sodium, hypermellose 3 CPS, colloidal silicon dioxide, talc and magnesium stearate.

In another embodiment, the capsule comprises from about 1% to about 90% w/w compound of formula (Ia); starch from about 2% to about 40%; microcrystalline cellulose from about 2% to about 90% w/w; mannitol from about 2% to 90% w/w; lactose monohydrate from about 2% to about 90% w/w; colloidal silicon dioxide from about 0.5% to about 5% w/w; talc from about 0.5 to about 5% w/w; magnesium stearate from about 0.5% to about 5% w/w based on the weight of the capsule.

The term “pharmaceutically acceptable” indicates that the material does not have properties that would cause one of skill in the art to avoid administration of the material to a patient, taking into consideration the disease or conditions to be treated and the respective route of administration. Further, the material is considered to be safe for administration in humans or animals.

The term “excipient” or “pharmaceutically acceptable excipient” refers to pharmacologically inactive substances that are added to a pharmaceutical preparation in addition to the active pharmaceutical ingredient. Excipients may take the function of vehicle, diluent, release, disintegration or dissolution modifying agent, absorption enhancer, stabilizer or a manufacturing aid among others. Excipients may include fillers (diluents), binders, disintegrating agents, lubricants, and glidants. Examples of excipient classes frequently used are listed below.

As used herein the term “diluent or filler” refers to substances that are used to dilute the active pharmaceutical ingredient prior to delivery. Diluents can also serve as stabilizers. Non-limiting examples of diluents include starch and its processed and co-processed denvertives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anyhydrous, magnesium oxide, maltodextrin, maltose, pullulan, sodium alginate, sodium bicarbonate, calcium silicate, calcium sulphate, cell and tribasic calcium phosphate or suitable combinations thereof.

As used herein the term “binder” refers to any pharmaceutically acceptable substance which can be used to bind the active and inert components together to maintain cohesive and discrete portions. Non-limiting examples of binders chitosan, hydrogenated castor oil, sodium alginate, carbomers, cellulose acetate phthalate, povidone, sugar, hydroxypropylmethyl-cellulose, hydroxypropylcellulose, starch, alginic acid, pregelatinized starch, acacia, tragakanth, ethylcellulose, acrylic and methacrylic acid co-polymers or suitable combinations thereof.

As used herein the term “disintegrant or disintegrating agents” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, agar, carboxymethyl cellulose calcium or sodium, colloidal silicon dioxide, chitosan, docusate sodium , hydroxyl propyl cellulose, magnesium aluminium silicate, maltose, methyl cellulose, polacrilin potassium, and alginic acid or suitable combinations thereof.

As used herein the term “lubricant” refers to an excipient, which is added to a powder blend to prevent the compacted powder mass from sticking to the equipment during the tableting or encapsulation process. It aids the ejection of the tablet form the dies, and can improve powder flow. Non-limiting examples of lubricants include magnesium stearate, stearic acid, silica, fats, zinc or sucrose or sodium or calcium stearate, castor oil, hydrogenated castor oil, . Polyethylene glycol and its derivatives, sodium stearyl fumarate, talc, or fatty acids including lauric acid, oleic acid, glyceryl behenate, glyceryl monostearate, and C₁-C₁₀ fatty acid or suitable combinations thereof.

As used herein the term “glidant” is intended to mean agents used in tablet and capsule formulations to improve flow-properties during tablet compression and to produce an anti-caking effect. Non-limiting examples of glidants include colloidal silicon dioxide, talc, fumed silica, starch, starch derivatives, and bentonite or suitable combinations thereof.

As used herein the term “plasticizer” refers to but not limited to polyols like polyethylene glycols, propyylene glycol, (glycerin),organic esters like phthalate esters(diethyl,dibutyl),dibutyl sebacete, citrate esters (triethyl, acetyl triethyl, acetyl tributyl), triacetin., Oils/glycerides like castor oil; acetylated monoglycerides, fractionated coconut oil or suitable combinations thereof.

As used herein the term “opacifier” refers to but not limited to titanium dioxide, talc, sunset yellow, tartrazine, erythrosine, iron oxide yellow, red and black, carmine, anthocyanins, allura Red AC, allura Red AC aluminum lake, indigotine, indigotine aluminum lake or suitable combinations thereof.

As used herein the term “film forming agents” refers to but not limited to hydroxypropyl methylcellulose, methylcellulose, ethylcellulose, hydroxypropyl cellulose, povidone, polydextrose, lactose, maltodextrin, acrylic polymer or suitable combinations thereof.

One or more solvents used in the formulation are selected from water, acetone, chloroform, dichloromethane, ethyl alcohol, ethyl acetate, methyl alcohol, isopropyl alcohol, N,N-dimethyl formamide and combinations thereof and other such materials known to those of ordinary skill in the art.

The pharmaceutical composition according to the present invention may be in the form of a tablet or capsule or a powder or a suspension in a liquid or an aerosol formulation or solutions, preferably in the form of a tablet or capsule.

The pharmaceutical composition of the present invention may be manufactured by any of the methods well-known in the art, such as by conventional mixing, dissolving, granulating, levigating, emulsifying, encapsulating, entrapping, or lyophilizing processes. As noted above, the compositions can include one or more pharmaceutically acceptable excipients that facilitate processing of active molecules into preparations for pharmaceutical use.

The present compositions may, if desired, be presented in a pack or dispenser device containing one or more unit dosage forms containing the active ingredient. Such a pack or device may, for example, comprise metal or plastic foil, such as a blister pack. Preferably the present composition can be packed in Alu/Alu blister or PVC-PVDC pack. Suitable conditions indicated on the label may include treatment of conditions, disorders, or diseases in which anemia is a major indication.

In another embodiment of the present invention, is described processes for the preparation of a pharmaceutical formulation of compounds of formula (la) or its pharmaceutically acceptable salts as described in examples 1-3.

The invention is further exemplified by the following non-limiting examples, which are illustrative representing the preferred modes of carrying out the invention. The invention’s scope is not limited to these specific embodiments only but should be read in conjunction with what is disclosed anywhere else in the specification together with those information and knowledge, which are within the general understanding of a person skilled in the art.

Example-1

Brief Manufacturing Procedure of oral tablet (uncoated)

| Sr. No. | Name of Ingredient | Quantity (% w/w) | | | | | |
|---|---|---|---|---|---|---|---|
| | | Formulation - 1 | Formulation - 2 | Formulation - 3 | Formulation - 4 | Formulation - 5 | Formulation - 6 |
| 1. | Compound (Ia) | 40.00 | 40.00 | 40.00 | 40.00 | 40.00 | 40.00 |
| 2. | Microcrystalline cellulose | 32.00 | 12.00 | -- | 9.60 | -- | 44 |
| 3. | Starch | -- | -- | 14.00 | 9.60 | 36.00 | - |
| 4. | Croscarmellose sodium | 8.00 | 6.40 | 4.00 | 4.00 | 6.40 | 5.00 |
| 5. | Lactose monohydrate | 14.00 | 37.20 | 36.00 | 29.20 | 9.60 | 5.00 |
| 6. | Hyperomellose | 4.00 | -- | -- | -- | -- | 4.00 |
| 7. | Polyvinyl Pyroliddone | -- | 2.40 | 4.00 | 4.00 | 4.80 | - |
| 8. | Collodial silicon dioxide | -- | -- | -- | 1.60 | 1.20 | - |
| 9. | Talc | 1.00 | 1.00 | 1.00 | 1.00 | 1.00 | 1.00 |
| 10. | Magnesium stearate | 1.00 | 1.00 | 1.00 | 1.00 | 1.00 | 1.00 |
| 11. | Purified Water | | | | | | |
| Total | | 100.00 | 100.00 | 100.00 | 100.00 | 100.00 | 100.00 |

Manufacturing Process:
1. All materials were sifted through appropriate sized sieves.
2. Intragranular material part (as per requirement based on composition) was transferred to RMG bowl and mixed for 5 minutes.
3. Wet granulations of mixed material was done with purified water keeping impeller ON followed by kneading of dough mass with both chopper and impeller running at high speed.
4. Wet mass was dried using fluid bed dryer at appropriate temperature until desired % LOD limit achieved.
5. After completion of drying, milling of dried granules was done using appropriate sized sieve.
6. Required quantity of colloidal silicon dioxide was blended with dried granules in blender for 3 minutes at 18 RPM.
7. Calculated amount of talc and magnesium stearate was added in blender and blended for 3 minutes at 18 RPM.

Prepared lubricated blend was compressed using tablet compression machine.

Example-2

Brief Manufacturing Procedure of oral tablet (uncoated)

| Sr. No. | Formulation composition | Formulation - 1 | Formulation - 2 | Formulation - 3 | Formulation - 4 | Formulation - 5 | Formulation - 6 | Formulation - 7 |
|---|---|---|---|---|---|---|---|---|
| | Ingredient | Quantity (% w/w) | | | | | | |
| Tablet core | | | | | | | | |
| 1. | Compound (Ia) | 38.83 | 38.83 | 38.83 | 38.83 | 38.8 | 38.83 | 38.83 |
| 2. | Microcrystalline cellulose | 51.81 | 31.07 | 11.65 | -- | 9.32 | -- | 42.72 |
| 3. | Starch | -- | -- | -- | 13.59 | 9.32 | 34.95 | - |
| 4. | Croscarmellose sodium | 2 | 7.77 | 6.21 | 3.88 | 3.88 | 6.21 | 4.8.5 |
| 5. | Lactose monohydrate | 1.5 | 13.59 | 36.12 | 34.95 | 28.35 | 9.32 | 4.85 |
| 6. | Hypromellose | 1 | 3.88 | -- | -- | -- | -- | 3.88 |
| 7. | Polyvinyl pyroliddone | -- | -- | 2.33 | 3.88 | 3.88 | 4.66 | |
| 8. | Collodial Silicon dioxide | -- | -- | -- | -- | 1.55 | 1.17 | |
| 9. | Talc | 0.97 | 0.97 | 0.97 | 0.97 | 0.97 | 0.97 | 0.97 |
| 10. | Magnesium stearate | 0.97 | 0.97 | 0.97 | 0.97 | 0.97 | 0.97 | 0.97 |
| 11. | Purified water | q.s. | q.s. | q.s. | q.s. | q.s. | q.s. | q.s. |
| Tablet coating | | | | | | | | |
| 12. | Hypromellose 3 cps | 1.94 | 1.94 | 1.94 | 1.94 | 1.94 | 1.94 | - |
| 13. | Opadry pink | - | - | - | - | - | - | 2.93 |
| 14. | Polyethylene glycols | 0.49 | 0.49 | 0.49 | 0.49 | 0.49 | 0.49 | - |
| 15. | Titanium dioxide | 0.49 | 0.49 | 0.49 | 0.49 | 0.49 | 0.49 | - |
| Total | | 100.00 | 100.00 | 100.00 | 100.00 | 100.00 | 100.00 | 100.00 |

Manufacturing Process:
1. All materials were sifted through appropriate sized sieves.
2. Intragranular material part (as per requirement based on composition) was transferred to RMG bowl and mixed for 5 minutes.
3. Wet granulations of mixed material was done with purified water keeping impeller ON followed by kneading of dough mass with both chopper and impeller running at high speed.
4. Wet mass was dried using fluid bed dryer at appropriate temperature until desired % LOD limit achieved.
5. After completion of drying, milling of dried granules was done using appropriate sized sieve.
6. Required quantity of colloidal silicon dioxide was blended with dried granules in blender for 3 minutes at 18 RPM.
7. Calculated amount of talc and magnesium stearate was added in blender and blended for 3 minutes at 18 RPM.
8. Prepared lubricated blend was compressed using tablet compression machine.
9. Prepared lubricated blend was compressed using tablet compression machine.
10. Coating of core tablets was done by spraying the coating dispersion in tablet coating machine to get desired tablet weight.

Example-3

Brief Manufacturing Procedure of oral capsule

| Sr. No. | Name of Ingredient | Quantity (% w/w) | | | | | | | |
|---|---|---|---|---|---|---|---|---|---|
| | | Formulation - 1 | Formulation - 2 | Formulation - 3 | Formulation - 4 | Formulation - 5 | Formulation - 6 | Formulation - 7 | Formulation - 8 |
| 1. | Compound (Ia) | 75.00 | 75.00 | 75.00 | 75.00 | 75.00 | 40.00 | 40.00 | 40.00 |
| 2. | Starch | -- | 13.75 | -- | 3.75 | -- | -- | -- | -- |
| 3. | Lactose monohydrate | 22.00 | -- | 15.00 | 7.50 | -- | 5.00 | 39.00 | 25.00 |
| 4. | Microcrystalline Cellulose | 0.00 | 10.00 | -- | 7.50 | -- | 44.00 | 10.00 | 24.00 |
| 5. | Croscarmellose sodium | -- | -- | -- | -- | -- | 5.00 | 3.00 | 8.00 |
| 6. | Hypermellose 3 cps | -- | -- | -- | -- | -- | 4.00 | 5.50 | 2.00 |
| 7. | Mannitol | -- | - | 7.50 | 3.75 | 22.50 | -- | -- | -- |
| 8. | Colloidal silicon dioxide | 1.25 | 1.25 | 1.25 | 1.25 | 1.25 | -- | -- | -- |
| 9. | Talc | 1.25 | | 0.63 | 1.25 | 0.63 | 1.00 | 1.00 | 0.50 |
| 10. | Magnesium Stearate | -- | -- | 0.63 | -- | 0.63 | 1.00 | 1.50 | 0.50 |
| 11. | Purified water | q.s. | q.s. | q.s. | q.s. | q.s. | q.s. | q.s. | q.s. |
| 12. | Empty hard gelatin capsule shell | -- | -- | -- | -- | -- | -- | -- | -- |
| Total | | 100.00 | 100.00 | 100.00 | 100.00 | 100.00 | 100.00 | 100.00 | 100.0 |

Manufacturing Process:
1. All materials were sifted through appropriate sized sieves.
2. Intragranular material part (as per requirement based on composition) was transferred to RMG bowl and mixed for 5 minutes.
3. Wet granulations of mixed material was done with purified water keeping impeller ON followed by kneading of dough mass with both chopper and impeller running at high speed.
4. Wet mass was dried using fluid bed dryer at appropriate temperature until desired % LOD limit achieved.
5. After completion of drying, milling of dried granules was done using appropriate sized sieve.
6. Required quantity of colloidal silicon dioxide was blended with dried granules in blender for 3 minutes at 18 RPM.
7. Calculated amount of talc and magnesium stearate was added in blender and blended for 3 minutes at 18 RPM.
8. Lubricated blend was filled into appropriate size hard gelatin capsule shell.

Stability studies are carried out at 30° C./65% RH, 30° C./75% RH, and at 40° C./75% RH. The stability data is given below:

Stability data of formulation-7 of Compound (Ia) 50 mg and 100 mg uncoated tablets
Compound (Ia) tablets 50 mg

| Test | Initial | 40° C./75%RH | | | 30° C./65%RH | |
|---|---|---|---|---|---|---|
| | | 1 month | 3 month | 6 month | 3 month | 6 month |
| Water content (%w/w) | 2.74 | 2.36 | 2.18 | 2.58 | 2.14 | 2.36 |
| Disintegration time (minutes) | 6.59 | 6.50 | 6.18 | 6.22 | 6.19 | 6.25 |
| Assay (%) | 100.52 | 100.31 | 101.74 | 99.93 | 99.42 | 100.84 |
| Total Impurity (%) | Not detected | Not detected | Not detected | Not detected | Not detected | Not detected |

Compound (Ia) tablets 100 mg

| Test | Initial | 40° C./75%RH | | 30° C./65%RH |
|---|---|---|---|---|
| | | 1 month | 3 month | 3 month |
| Water content (%w/w) | 3.37 | 3.21 | 4.05 | 3.70 |
| Disintegration time (minutes) | 2.55 | 2.59 | 3.11 | 3.04 |
| Assay (%) | 99.14 | 98.76 | 98.41 | 98.72 |
| Total Impurity (%) | 0.34 | 0.43 | 0.40 | 0.38 |

Stability data of formitiatioit-6 of compound (Ia) tablets 50 mg and 100 mg coated tablets
Compound (Ia) tablets 50 mg (coated tablets)

| Test | Initial | 40°±2° C. & 75± 5% RH | | | 30°±2° C. & 75± 5%RH | |
|---|---|---|---|---|---|---|
| | | 1 month | 3 month | 6 month | 3 month | 6 month |
| Water content (%w/w) | 3.30 | 3.90 | 5.40 | 4.94 | 5.00 | 5.16 |
| Assay (%) | 103.00 | 101.90 | 100.70 | 102.80 | 102.60 | 102.50 |
| Total Impurity (%) | 0.16 | 0.13 | 0.08 | 0.11 | 0.09 | 0.11 |

Compound (Ia) 100 mg (coated tablets)

| Test | Initial | 40°±2° C. & 75± 5%RH | | | 30°±2° C. & 75± 5%RH | |
|---|---|---|---|---|---|---|
| | | 1 month | 3 month | 6 month | 3 month | 6 month |
| Water content (%w/w) | 3.20 | 4.00 | 5.20 | 4.60 | 4.50 | 4.80 |
| Assay (%) | 102.90 | 101.40 | 101.40 | 102.20 | 101.20 | 102.80 |
| Total Impurity (%) | 0.15 | 0.13 | 0.09 | 0.12 | 0.09 | 0.08 |

Stability data of formulation-7 of Compound (Ia) tablets 25 mg, 50 mg, 100 mg and 200 mg Uncoated tablets
Compound (Ia) tablets 25 mg (uncoated tablets)

| Test | Initial | 40°±2° C. & 75± 5%RH | | | 30°±2° C. & 75± 5%RH | |
|---|---|---|---|---|---|---|
| | | 1 M | 3 M | 6 M | 3 M | 6 M |
| Water content (% w/w) | 3.16 | 2.56 | 2.98 | 2.90 | 2.98 | 2.87 |
| Disintegration time (Min: Sec) | 04:25 | 05:27 | 05:04 | 05:11 | 05:39 | 05:12 |
| Assay (%) | 99.5 | 99.6 | 98.7 | 99.6 | 99.4 | 99.3 |
| Total Impurity (%) | 0.00 | 0.00 | 0.00 | 0.00 | 0.00 | 0.00 |

Compound (Ia) tablets 50 mg (uncoated tablets)

| Test | Initial | 40°±2° C. & 75± 5%RH | | | 30°±2° C. & 75± 5%RH | |
|---|---|---|---|---|---|---|
| | | 1 M | 3 M | 6 M | 3 M | 6 M |
| Water content (% w/w) | 3.08 | 2.61 | 2.46 | 2.94 | 2.41 | 3.17 |
| Disintegration time (Min: Sec) | 05:33 | 05:51 | 05:53 | 05:56 | 05:44 | 05:49 |
| Assay (%) | 100.0 | 98.2 | 99.7 | 98.8 | 100.7 | 98.5 |
| Total Impurity (%) | 0.00 | 0.00 | 0.00 | 0.00 | 0.00 | 0.00 |

Compound (Ia) tablets 100 (uncoated tablets)

| Test | Initial | 40°±2° C. & 75± 5%RH | | | 30°±2° C. & 75± 5%RH | |
|---|---|---|---|---|---|---|
| | | 1 M | 3 M | 6 M | 3 M | 6 M |
| Water content (% w/w) | 3.05 | 2.03 | 2.91 | 2.46 | 3.42 | 2.46 |
| Disintegration time (Min: Sec) | 02:16 | 02:10 | 01:58 | 02:01 | 02:04 | 02:06 |
| Assay (%) | 103.3 0 | 103.7 0 | 103.2 0 | 103.3 0 | 102.70 | 104.30 |
| Total Impurity (%) | 0.1 | 0.17 | 0.16 | 0.17 | 0.16 | 0.16 |

Compound (Ia) tablets 200 mg (uncoated tablets)

| Test | Initial | 40°±2° C. & 75± 5%RH | | | 30°±2° C. & 75± 5%RH | |
|---|---|---|---|---|---|---|
| | | 1 M | 3 M | 6 M | 3 M | 6 M |
| Water content (% w/w) | 2.49 | 1.36 | 3.57 | 1.92 | 2.67 | 1.75 |
| Disintegration time (Min: Sec) | 03:39 | 03:10 | 03:49 | 04:30 | 03:55 | 04:37 |
| Assay (%) | 100.6 0 | 98.80 | 102.2 0 | 99.0 0 | 99.40 | 98.90 |
| Total Impurity (%) | 0.12 | 0.17 | 0.16 | 0.17 | 0.16 | 0.17 |

The above stability data shows that the formulations are stable and the compound of formula (Ia) is effectively stabilized so that it may be used in clinical trials and subsequently as a commercial product.

1. A parmaceutical composition comprising compound of formula (Ia) 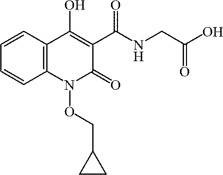 [SMILES: O=C(O)CNC(=O)c1c(O)c2ccccc2n(OCC2CC2)c1=O]
or its pharmaceutically acceptable salts and one or more pharmaceutical excipients.

2. The pharmaceutical composition as claimed in claim 1, wherein pharmaceutically acceptable excipients are selected form disintegrant, glidant, lubricant, diluent or filler, film forming agents, coating materials, binders, opacifier, plasticizers and solvents.

3. The pharmaceutical composition as claimed in claim 2, wherein the disintegrant is selected from maize starch, sodium starch glycolate, croscarmellose sodium, crospovidone, microcrystalline cellulose, modified corn starch, sodium carboxymethyl starch, povidone, pregelatinized starch, agar, carboxymethyl cellulose calcium or sodium, colloidal silicon dioxide, chitosan, docusate sodium , hydroxyl propyl cellulose, magnesium aluminium silicate, maltose, methyl cellulose, polacrilin potassium, and alginic acid or suitable combinations thereof.

4. The pharmaceutical composition as claimed in claim 2, wherein the glidants is selected from colloidal silicon dioxide, talc, fumed silica, starch, starch derivatives, and bentonite or suitable combinations thereof.

5. The pharmaceutical composition as claimed in claim 2, wherein the diluent or filler is selected from starch and its processed and co-processed derivertives, saccharides, di saccharides, sucrose, lactose, polysaccharides, cellulose, cellulose ethers, cellulose acetate, hydroxypropyl cellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di-basic calcium phosphate dihydrate, mannitol lactose anyhydrous, magnesium oxide, maltodextrin, maltose, pullulan, sodium alginate, sodium bicarbonate, calcium silicate, calcium sulphate, cell and tribasic calcium phosphate or suitable combinations thereof.

6. The pharmaceutical composition as claimed in claim 2, wherein the lubricant is selected from magnesium stearate, stearic acid, silica, fats, zinc or sucrose or sodium or calcium stearate, castor oil, hydrogenated castor oil, . Polyethylene glycol and its derivatives, sodium stearyl fumarate, talc, or fatty acids including lauric acid, oleic acid, glyceryl behenate, glyceryl monostearate, and C /C10 fatty acid or suitable combinations thereof.

7. The pharmaceutical composition as claimed in claim 2, wherein the plasticizers is selected from polyols like polyethylene glycols PEG, propylene glycol, glycerol (glycerin), organic esters like phthalate esters (diethyl, dibutyl), dibutyl sebacete, citrate esters (triethyl, acetyl triethyl, acetyl tributyl), triacetin., Oils/glycerides like castor oil; acetylated monoglycerides, fractionated coconut oil or suitable combinations thereof.

8. The pharmaceutical composition as claimed in claim 2, wherein the opacifier is selected from titanium dioxide, talc, sunset yellow, Tartrazine, Erythrosine, iron oxide yellow, red and black, Carmine, Anthocyanins. Allura Red AC, Allura Red AC aluminum lake, Indigotine, Indigotine aluminum lake or suitable combinations thereof.

9. The pharmaceutical composition as claimed in claim 1, wherein the film forming agent is selected from hydroxypropyl methylcellulose, methylcellulose, ethylcellulose, hydroxypropyl cellulose, povidone, polydextrose, lactose, maltodextrin, acrylic polymer or suitable combinations thereof.

10. The pharmaceutical composition as claimed in claim 1 is in the form of a tablet or a caplet or a capsule or a powder or a suspension in a liquid or an aerosol formulation or solutions, preferably in the form of a tablet or capsule.

11. The pharmaceutical composition as claimed in claim 1, wherein the compound of formula (la) or its pharmaceutically acceptable salts have D₉₀ value of not more than 450 microns.

12. The pharmaceutical composition as claimed in claim 1 as an uncoated tablet formulation comprises compound of formula (la) and one or more pharmaceutically acceptable excipients selected from microcrystalline cellulose, starch, croscarmellose sodium, lactose monohydrate, hypromellose, polyvinyl pyrrolidone, colloidal silicon dioxide, talc and magnesium stearate.

13. The pharmaceutical composition as claimed in claim 11, wherein uncoated tablet comprises from about 1% to about 90% w/w compound of formula (la); Microcrystalline cellulose from about 2% to about 90% w/w; Croscarmellose Sodium from about 0.5% to 10 % w/w; Lactose Monohydrate from about 2% to about 90% w/w; Hypromellose 3 cps from about 0.5% to about 10% w/w; Talc from about 0.5 % to about 3% w/w; Magnesium Stearate from about 0.5% to about 5% w/w of the total composition, polyvinyl pyrolidone from about 0.5% to about 10% w/w; starch from about 1% to about 20% w/w based on the weight of uncoated tablet.

14. The pharmaceutical composition as claimed in claim 1 as a coated tablet formulation comprising a tablet core and a coating comprises compound of formula (la) and one or more pharmaceutically acceptable excipients selected from microcrystalline cellulose, starch, croscarmellose sodium, lactose monohydrate, hypromellose, polyvinyl pyrrolidone, colloidal silicon dioxide, talc, magnesium stearate, polyethylene glycols, titanium dioxide or suitable coating ready materials selected from Opadry.

15. The pharmaceutical composition as claimed in claim 14, wherein the coating is present in the tablet in an amount is from about 0.5% to about 5% w/w of hypromellose 3Cps; polyethylene glycol from about 0.25% to about 1.0% w/w; titanium dioxide from about 0.25% to about 2.0% w/w; opadry pink from about 0.5% to about 5% based on the weight of the tablet core.

16. The pharmaceutical composition as claimed in claim 14, wherein the tablet core comprises from about 1% to about 90% w/w compound of formula (Ia); microcrystalline cellulose from about 2% to about 90% w/w; croscarmellose sodium from about 0.5% to 10% w/w; lactose monohydrate from about 2% to about 90% w/w; hypromellose 3 cps from about 0.5% to about 10% w/w; talc from about 0.5 to about 5% w/w; magnesium stearate from about 0.5% to about 3%of based on the weight of the coated tablet.

17. The pharmaceutical composition as claimed in claim 1 as an oral capsule formulation wherein capsule is either capsule fill or capsule shell comprises compound of formula (Ia) and one or more pharmaceutically acceptable excipients selected from microcrystalline cellulose, starch, mannitol, lactose monohydrate, croscarmellose sodium, hypermellose 3 CPS, colloidal silicon dioxide, talc and magnesium stearate.

18. The pharmaceutical composition as claimed in claim 17, wherein capsule is either capsule fill or capsule shell comprises from about 1% to about 90% w/w compound of formula (la); starch from about 2% to about 40%; microcrystalline cellulose from about 2% to about 90% w/w; mannitol from about 2% to 90% w/w; lactose monohydrate from about 2% to about 90% w/w; colloidal silicon dioxide from about 0.5% to about 5% w/w; talc from about 0.5 to about 5% w/w; magnesium stearate from about 0.5% to about 5% w/w based on the weight of the capsule.

19. The pharmaceutical composition as claimed in claim 1 is prepared as described in examples 1 to 3.

20. A method for treating, pretreating, or delaying onset or progression of anemia, comprising administering to a patient in need thereof, a pharmaceutical composition of claim 1.
